(19) United States
(12) Patent Application Publication
(10) Pub. No.: US 2007/0016029 A1
(43) Pub. Date: Jan. 18, 2007

(54) Physiology workstation with real-time fluoroscopy and ultrasound imaging

(75) Inventors: Brenda Donaldson, Harrison Township, MI (US); Israel Raz, Highland Park, IL (US); Sachin Vadodaria, Fox Point, WI (US)

Correspondence Address: THE SMALL PATENT LAW GROUP LLP, 611 OLIVE STREET, SUITE 1611, ST. LOUIS, MO, 63101, US

(73) Assignee: General Electric Company

(21) Appl. No.: 11/182,910

(22) Filed: Jul. 15, 2005

(51) Int. Cl. A61B 8/00

(52) U.S. Cl. 600/437

(57) ABSTRACT

A physiology workstation is provided that comprises an physiology input configured to receive physiology signals from at least one of an intracardiac (IC) catheter inserted in a subject and surface ECG leads provided on the subject. The physiology signals are obtained during a procedure. A video input is configured to receive image frames, in real-time during the procedure. The image frames contain diagnostic information representative of data samples obtained from the subject during the procedure. A control module controls physiology operations based on user inputs. A display module is controlled by the physiology control module. The display module displays the physiology signals and the image frames simultaneously, in real-time, during the procedure. Optionally, the workstation may include a video processor module that formats the physiology signals into a display format. The video processor module may include an video processor and an external video processor that receive and control display of the physiology signals and image frames, respectively. The image frames may include at least one of ultrasound images obtained from a surface ultrasound probe, intravenous ultrasound images obtained from an ultrasound catheter and fluoroscopy images obtained from a fluoroscopy system.

### BACKGROUND OF THE INVENTION

Embodiments of the present invention generally relate to electrophysiology (EP) workstations, hemo-dynamic (HD) workstations, fluoroscopy workstations and ultrasound imaging workstations. More particularly, embodiments of the present invention relate to providing a physiology workstation (e.g., EP or HD workstation) with real-time fluoroscopy imaging, ultrasound imaging and other diagnostic imaging modality.

EP, HD and ablation procedures are complex and sensitive procedures, and as such, utilize numerous diagnostic and therapeutic systems. Generally, EP, HD and ablation procedures are carried out in a procedure room including, among other things, EP catheters, HD catheters and patient sensors joined to an EP or HD workstation. The procedure room also includes a fluoroscopy system, a diagnostic ultrasound system, a patient monitoring device and an ablation system. A monitoring room and a control room may be located adjacent to the procedure room. The EP or HD workstation and a stimulator may be located in the control room. Alternatively, when remote monitoring rooms are not used, the EP or HD workstation and stimulator are provided in the procedure room in a corner or monitoring area. An example of a conventional HD workstation is the Mac-Lab® Hemodynamic Monitoring system offered by G.E. Healthcare. An example of a conventional EP workstation Is the Cardio Lab® EP Lab Monitoring system offered by G.E. Healthcare.

Conventional EP and HD workstations include monitors that present information related directly to the EP or HD study, such as EP or HD signals, case logs, patient information and the like. The diagnostic imaging systems (fluoroscopy, ultrasound and the like) are provided in the procedure room and are operated as stand-alone systems. For example, conventional fluoroscopy systems utilize one or two monitors provided on the fluoroscopy device located in the procedure room. The fluoro-monitors present fluoroscopy images to the procedure team to facilitate and monitor catheter placement and operation. Similarly, conventional ultrasound systems are constructed as stand-alone, independent units having a monitor and user interfaces on the system. The ultrasound system is positioned in the procedure room and operated by the procedure team. Images obtained by the ultrasound system are provided on the monitor mounted to the ultrasound system. The imaging capabilities of these various systems are independent and physically remote from one another.

Conventional EP and HD workstations and diagnostic systems suffer from various disadvantages, that are addressed by various embodiments of the present invention.

### BRIEF DESCRIPTION OF THE INVENTION

In accordance with one embodiment, a physiology workstation is provided that comprises a physiology input configured to receive physiology signals from at least one of an intracardiac (IC) catheter inserted in a subject, a hemodynamic catheter inserted in a subject, and surface ECG leads provided on the subject. The physiology signals are obtained during a procedure. A video input is configured to receive image frames, in real-time during the procedure. The image frames contain diagnostic information representative of data samples obtained from the subject during the procedure. A control module controls workstation operations based on user inputs. A display module is controlled by the EP control module. The display module displays the physiology signals and the image frames simultaneously, in real-time, during the procedure.

Optionally, the physiology workstation may include a video processor module that formats the physiology signals into a display format. The video processor module may include a physiology signal video processor and an external video processor that receive and control display of the physiology signals and image frames, respectively. The image frames may include at least one of ultrasound images obtained from a surface ultrasound probe, or intravenous, intraarterial or transesophageal ultrasound images obtained from an ultrasound probe and fluoroscopy images obtained from a fluoroscopy system.

In accordance with another embodiment, a method is provided for managing a physiology workstation. The method comprises receiving, at a physiology workstation, physiology signals from at least one of an intracardiac (IC) catheter inserted in a subject, a hemodynamic (HD) catheter inserted in a subject and surface ECG leads provided on the subject, the physiology signals being obtained during a procedure. The method also includes receiving, at the workstation, image frames, in real-time during the procedure, the image frames containing diagnostic information representative of data samples obtained from the subject during the procedure. Physiology operations are controlled based on user inputs and the physiology signals and the image frames are displayed simultaneously, in real-time, during the procedure at the workstation.

Optionally, the method may include synchronizing the physiology signals and image frames and displaying the physiology signals and image frames in a synchronized manner based on a cardiac cycle of the subject. Alternatively, the physiology signals and image frames may be displayed in a non-synchronized manner.

### BRIEF DESCRIPTION OF THE DRAWINGS

FIG. 1 illustrates a block diagram of an image management system formed in accordance with an embodiment of the present invention.

FIG. 2 illustrates a block diagram of an image management system formed in accordance with an alternative embodiment of the present invention.

FIG. 3 illustrates a pictorial representation of an image management flow carried out in accordance with an embodiment of the present invention.

FIG. 4 illustrates a screen shot of an exemplary monitor layout presented in accordance with an embodiment of the present invention.

FIG. 5 illustrates a block diagram of an image management system formed in accordance with an alternative embodiment of the present invention.

FIG. 6 illustrates a screen shot of an exemplary window on an HD workstation monitor presented in accordance with the an embodiment of the present invention.

FIG. 7 illustrates a block diagram of an alternative embodiment in which remote control is provided for various systems and devices presented in accordance with the an embodiment of the present invention.

FIG. 8 illustrates an exemplary remote device user interface 708 constructed one to substantially resemble the keyboard of an ultrasound system presented in accordance with the an embodiment of the present invention.

FIG. 9 illustrates a processing sequence that may be carried out by the physiology workstation 206 presented in accordance with the an embodiment of the present invention.

FIG. 10 illustrate an image acquired by an echocardiography (ICE) catheter presented in accordance with the an embodiment of the present invention.

FIGS. 11A and 11B illustrates an ultrasound image obtained by an ICE catheter located on the right side of the inter-atrial septum positioned to image the left pulmonary veins presented in accordance with the an embodiment of the present invention.

FIGS. 12A and 12B represent ultrasound images obtained by an ICE catheter presented in accordance with the an embodiment of the present invention.

### DETAILED DESCRIPTION OF THE INVENTION

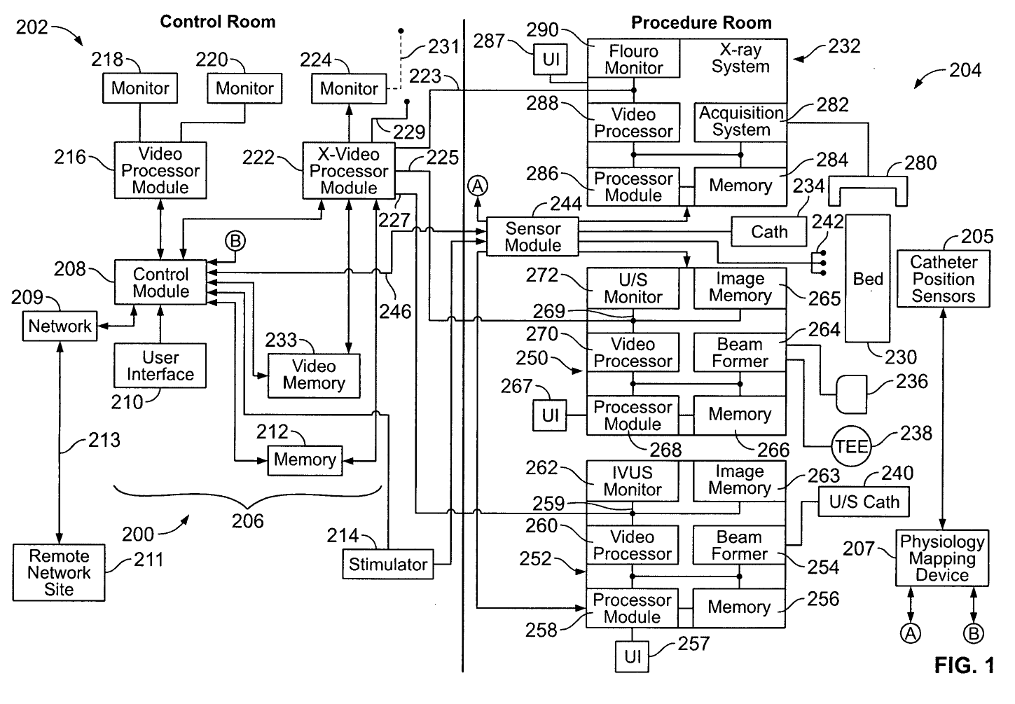

FIG. 1 illustrates an image management system 200 formed in accordance with an embodiment of the present invention. The image management system 200 may be distributed between a control room 202 and procedure room 204 or, alternatively, may be all located in the procedure room 204. Thus the image management system 200 may be located entirely in the procedure room 204. A physiology workstation 206 (e.g., EP or HD workstation) is provided to control and coordinate EP or HD procedures, ablation procedures and the like. The physiology workstation 206 includes a control module 208 that is controlled by an operator through user interface 210. Memory 212 stores various information as will be explained below in more detail. A stimulator 214 is provided to generate stimulus signals delivered to the patient in the procedure room 204. A physiology video processor module 216 communicates with the control module 208 and controls monitors 218 and 220. An external video processor module 222 is also provided within the workstation 206. The external video processor module 222 communicates with control module 208 and controls a real-time imaging monitor 224. Optionally, the physiology and external video processor modules may be combined as a single module and/or may implemented utilizing a single or parallel processors.

A physiology mapping device 207 is provided in the procedure room 204 and is joined to the workstation 206 over link B and to the sensor module 244 over link A. The physiology mapping device 207 communicates with catheter position sensors 205 to monitor the position of EP, HD and/or mapping catheters, while being positioned within the heart. Examples of a conventional EP mapping device 207 are the LocaLisa® intra-cardiac navigation system offered by Medtronic, Minneapolis, Minn., and the CARTO® system by Biosense Webster.

The workstation 206 integrates, among other things, real-time EP and HD information, real-time intracardiac (IC) echography, transesophageal ultrasound, transthoracic ultrasound, fluoroscopic images, EP mapping data and pre-surgery planning CT & MR images. The workstation 206 offers integrated monitoring and review of EP, HD, patient, and mapping information as well as stored and real-time diagnostic images, ECG signals and IC signals.

The procedure room 204 includes a patient bed 214 to hold the patient during pre-procedure intracardiac mapping and during EP, HD and ablation procedures. A fluoroscopy system 232 is provided proximate patient bed 214 to obtain fluoroscopic images of the region of interest while the doctor is conducting mapping or a procedure. EP or HD catheters 234, ultrasound probes 236, 238 and an ultrasound probe 240 are provided for use throughout the procedure. EP or HD catheter 234 performs sensing and stimulating functions. An ablation catheter (not shown) may represent an RF ablation catheter, a laser ablation catheter or a cryogenic ablation catheter. HD catheters may represent open lumen catheters that measure pressure.

The ultrasound catheter 240 and ultrasound probes 236, 238 are configured to obtain ultrasound images of the region of interest, as well as images that indicate directly the position and placement of other instruments, devices and catheters, such as a defibrillator or pacemaker lead, catheter 234, an ablation catheter and the like relative to the region of interest. Surface ECG leads 212 are provided and attached to the patient to obtain surface ECG information. The surface ECG leads 212 and the catheters 234 are joined to the sensor module 244 which amplifies and/or pre-conditions signals sensed by the surface ECG leads 212 and catheters 234 prior to transmitting the sensed signals over communications link 246. When stimulus pulses are to be delivered to the patient, the stimulus signals are passed either around or through the sensor module 244 to the corresponding catheters 234. An ablation source and controller (not shown) controls operation of the ablation catheter and provides ablation-related data to the workstation 206. The ablation technique may be cryosurgical, radio frequency, high intensity focused ultrasound, microwave, laser and the like.

An ultrasound system 250 and an intravascular ultrasound (IVUS) system 252 are joined to, and control, the ultrasound probes 236, 238 and catheter 240. The ultrasound catheter 240 may generally represent an intravascular ultrasound (IVUS) catheter, in that the catheter 240 and IVUS system 252 may be used to perform diagnostic ultrasound examination of any and all portions of a subjects vascular structure, including but not limited to, the cardiac structure, peripheral veins, peripheral arteries and the like. One exemplary application of an IVUS system 252 is to perform intracardiac echocardiography (ICE), in which the catheter 240 is utilized in an intra-cardiac examination. An user interface 257 permits an operator to control operation of the IVUS system 252, and to enter modes, parameters and settings for the IVUS system 252. The IVUS system 252 includes a beamformer 254 that is responsible for transmit and receive beamforming operations. The link between the beamformer 254 and ultrasound catheter 240 may comprise individual channels associated with each transducer element within the transducer head of the ultrasound catheter 240. The beamformer 254 controls the phase and amplitude of each transmit signal delivered over the link to induce a transmit or firing operation by the ultrasound catheter 240. Reflected echoes are received at the ultrasound catheter 240 and delivered to the beamformer 254 as analog or digital signals representative of the detected echo information at each individual transducer element.

The beamformer 254 may include a demodulator and filters (or a processor programmed) to demodulate and filter the received echo signals. The beamformer 254 generates RF signals from echo signals and performs RF processing to produce digital base-band I and Q data pairs formed from the RF signals associated with acquired data samples. The I, Q data pairs are derived from the reflected ultrasound signals from respective focal zones of the transmitted beams. The I and Q data pairs are filtered, such as in FIR filters that are programmed with filter co-effecients to pass a band of frequencies centered at a desired fundamental frequency of the transmit waveform or at harmonic or sub-harmonic frequencies of the transmit signal's fundamental frequency. An I, Q data pair corresponds to each data sample within the region of interest. The beamformer 254 may pass the I, Q data pairs to memory 256, or directly to processor module 258.

The I, Q data pairs are processed by mode-related modules (e.g., B-mode, color Doppler, power Doppler, M-mode, spectral Doppler anatomical M-mode, strain, strain rate, and the like) of the processor module 258 to form 2D or 3D data sets of image frames, volumetric data sets and the like. For example, the processor module 258 may generate B-mode, color Doppler, power Doppler, M-mode, anatomical M-mode, strain, strain rate, spectral Doppler image frames and combinations thereof, and the like. The image frames are stored in memory 256. The processor module 258 may record, with each image frame, timing information indicating a time at which the image frame was acquired. The processor module 258 may also include a scan conversion module to perform scan conversion operations to convert the image frames from Polar to Cartesian coordinates. A video processor module 260 reads the image frames from memory 256 and displays the image frames on the IVUS monitor 262 in real time during the procedure is being carried out on the patient. Optionally, the video processor module 260 may store the image frames in an image memory 263, from which the images are read and displayed on IVUS monitor 262.

A video link 259 is maintained between the video processor 260, image memory 263 and IVUS monitor 262. The IVUS system 252 includes a video output (e.g., a VGA output) that is connected to a video link 227 (e.g., a VGA cable). The video link 227 conveys to the physiology workstation 206 the identical video signals as presented to the IVUS monitor 262.

The ultrasound system 250 includes a transmitter (within beamformer 264) which drives ultrasound probes 236, 238. An user interface 267 permits an operator to control the operation of, and enter modes, parameters and settings for, the ultrasound (U/S) system 250. The ultrasound probes 236, 238 include transducer arrays that emit pulsed ultrasonic signals into a region of interest. The probes 236, 238 may be moved over the region of interest 2D or 3D volumetrically in order to acquire image information in scan planes of the region of interest. The probes 236, 238 may conform to one of many geometries, as examples, transesophageal, a 1D, 1.5D, 1.75D, or 2D probe. Structures in the region of interest (e.g., a heart, blood cells, muscular tissue, and the like) back-scatter the ultrasonic signals. The resultant echoes return to the transducers. In response, the transducers generate electrical signals that the receiver receives and forwards to the beamformer 264.

The beamformer 264 processes the signals for steering, focusing, amplification, and the like. The beamformer 264 generates RF signals based on the received echoes. The beamformer 264 also filters and demodulates the RF signals to form in-phase and quadrature (I/Q) data pairs representative of the echo signals from data samples. The RF or I/Q signal data may then be routed to the memory 266 for storage or directly to the processor module 268.

The processor module 268 acquires ultrasound information (i.e., the RF signal data or IQ data pairs) from memory 266 and prepares frames of ultrasound information (e.g., graphical images) for storage or display. The processor module 268 may record, with each image frame or volume, timing information indicating a time at which the image frame was acquired The processor module 268 provides the ultrasound information to the video processor 270. The video processor 270 stores image frame data in the image memory 265 and outputs the video signals that drive the monitor 272. The monitor 272 may be, as examples, a CRT or LCD monitor, hardcopy device, or the like.

The processor module 268 executes instructions out of the program memory 266. The memory 266 stores, for example, an operating system for the ultrasound system 250, image processing programs, and the like. In general, the processor module 268 performs any selected processing operation available on the acquired ultrasound information chosen from the configured ultrasound modalities present in the ultrasound imaging system 250. The processor module 268 may process in real-time acquired ultrasound information during a scanning session as the echo signals are received. Additionally or alternatively, the ultrasound information may be stored temporarily in the memory 266 during a scanning session and processed in less than real-time in a live or off-line operation.

The ultrasound system 250 may acquire ultrasound information at a selected frame rate (e.g., 12.5, 15, 25, 30, 50 or 60 frames per second) and display those frames at the same or different frame rate on the monitor 272. The memory 266 shown in FIG. 1 may store processed frames that are not scheduled for immediate display. For example, the memory 266 may be sized to store several seconds or more of image frames. In one embodiment, the ultrasound system 250 stores the image frames with triggering information (e.g., ECG signal or respiratory signal) so that the ultrasound system 250 can present looping image sequences on the monitor 272, synchronized to selected events in the region of interest (e.g., heart cycle or breathing cycle).

In addition or alternatively, the ultrasound system 250 may scan a volume from the region of interest. To that end, the probes 236, 238 may be used in conjunction with techniques including 3D scanning, real-time 3D imaging, volume scanning, 2D scanning with transducers having positioning sensors, freehand scanning using a Voxel correlation technique, 2D or matrix array transducers and the like.

When the probes 236, 238 move, as examples, along a linear or arcuate path, it scans the region of interest. At each linear or arcuate position, the probes 236, 238 obtain scan planes from the region of interest. The scan planes are collected to cover a selected thickness, for example, by collecting adjacent scan planes. The scan planes are stored in the memory 266, and then passed to a volume scan converter in the processor module 268. In some embodiments, the probes 236, 238 may obtain lines instead of the scan planes, and the memory may store lines obtained by the probe rather than the scan planes.

A volume scan converter module in the processor module 268 receives a slice thickness setting from a control input at user interface 267, that an operator adjusts to choose the thickness of a slice to be created from the scan planes. The volume scan converter module in the processor module 268 creates a data slice from multiple adjacent scan planes. The number of adjacent scan planes that form each data slice is dependent upon the thickness selected by the slice thickness control input. The data slice is stored in memory 266 for access by the volume rendering processor in the processor module 268. The volume rendering processor module in the processor module 268, in conjunction with image display programs in the memory 266, performs volume rendering upon the data slice. The output of the volume rendering processor module passes to the video processor 270 and monitor 272.

A video link 269 is maintained between video processor module 270, image memory 265 and U/S monitor 272. the U/S system 250 includes a video output (e.g., VGA output) that is connected to a video link 225 (e.g., a VGA cable). The video link 225 conveys to the physiology workstation 206 the identical video signals as presented to the U/S monitor 272.

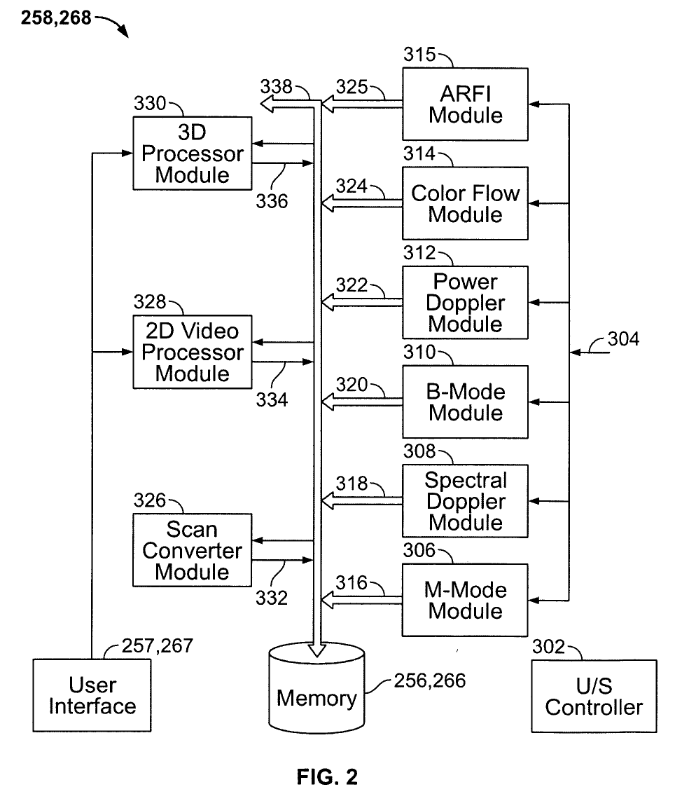

The processor module 258 in the IVUS system 252 and the processor module 268 in the ultrasound system 250 may also receive hemodynamic, inter-cardiac and/or surface ECG signals from the sensor module 244, surface leads 242 and catheter 234. Optionally, the processor modules 258 and 268 may receive respiratory signals corresponding to the breathing cycle of the patient. The processor modules 258 and 268 utilize the IC signals, HD signals, ECG signals and/or respiratory signals to derive timing information that is tagged to each ultrasound image frame generated by the scanned converter 326 (FIG. 2). In one mode of operation, the ultrasound system 250 displays sequences of images captured by the probes 236, 238. One or more of the images may be displayed in synchronism with an event trigger determined by in the processor module 268.

Optionally, the IVUS system 252 and/or the ultrasound system 250 may be operated in an acoustic radiation force imaging (ARFI) mode. ARFI allows examination of the functionality of tissue subsets, such as in the heart, organs, tissue, vasculature and the like. ARFI is a phenomenon associated with the propagation of acoustic waves through a dissipative medium. It is caused by a transfer of momentum from the wave to the medium, arising either from absorption or reflection of the wave. This momentum transfer results in the application of a force in the direction of wave propagation. The magnitude of this force is dependent upon both the tissue properties and the acoustic beam parameters. The duration of the force application is determined by the temporal profile of the acoustic wave. ARFI images the response of tissue to acoustic radiation force for the purpose of characterizing the mechanical properties of the tissue. When the duration of the radiation force is short (less than 1 millisecond), the tissue mechanical impulse response can be observed. ARFI imaging has many potential clinical applications, including: detecting and characterizing a wide variety of soft tissue lesions, and identifying and characterizing atherosclerosis, plaque, and thromboses.

The procedure room 204 may include various equipment and systems, such as an x-ray system 232 that controls a rotating support arm 280. The modes, parameters and other settings of the x-ray system 232 are entered and controlled from the user interface 287. The support arm 280 includes a x-ray source and a x-ray detector on opposite ends thereof. The x-ray detector may represent an image intensifier, a flat panel detector, a change coupled device and the like. The x-ray detector provides fluoroscopy data to a data acquisition system 282 which stores the x-ray data in memory 284. A processor module 286 processes the x-ray data to generate x-ray images that may be stored in memory 284 or passed directly to video processor module 288. The processor module 286 also receives HD, IC and/or ECG signals from the sensor module 244. The processor module 286 enters timing information with each image frame representing the time at which the frame was acquired. The video processor 288 may include a frame grabber which obtains single x-ray images from the memory 284 and controls presentation of the x-ray images on the monitor 290.

In each of the x-ray system 232, IVUS system 252 and U/S system 250, the timing information may be derived from the time of day, or from a reference clock. Alternatively, the various processors may have synchronized clocks which result in all the various systems being synchronized to the identical spot in the cardiac cycle. Alternatively, the timing information may be associated with the cardiac cycle of the patient which is determined by the EP signals provided from the sensor module 244.

The workstation 206 includes a physiology control module 208 which is configured to receive and transmit a variety of signals and data that are conveyed to and from the patient over leads, cables, catheters and the like. Examples of signals that may be received by the control module 208 include intercardiac (IC) signals and/or hemodynamic signals from catheters 234, patient monitoring signals (e.g., from a blood pressure cuff, SPO2 monitor, temperature monitor, CO2 levels and the like), ECG signals from surface ECG leads 212.

When separate rooms are used, the link 246 extends from the workstation 206, through the wall or other divider separating the control and procedure rooms 202 and 204, into the procedure room. Alternatively, all equipment may be in the same room. The link 246 conveys, among other things, IC signals, hemodynamic signals, patient monitoring signals, surface ECG signals and pressure signals. The content and nature of the information conveyed over the link 246 is explained below in more detail. In one embodiment, the link 246 is comprised of physical connections (e.g. analog lines, digital lines, coaxial cables, Ethernet data cables and the like or any combination thereof). Optionally, the link 246 may be all or partially wireless (e.g., an RF link).

The workstation 206 is used in an EP or HD study, such as to provide a detailed evaluation of the heart's electrical system. During an EP or HD study, typically 3-5 catheters may be used. Each EP catheter 234 includes platinum electrodes spaced near the tip of the catheter, where such electrodes have the ability to record electrical signals from inside the heart as well as deliver stimulus pulses to the heart from different locations, such as to pace the heart. The workstation 206 evaluates normal and abnormal conductions and rhythms. The protocol used during the EP study may vary from site to site or procedure to procedure (e.g. corrected sinus node recovery time, AV Wenckebach and the like). Typically, HD catheters 234 have an open lumen to monitor pressure.

The control module 208 communicates directly with an external stimulator 214, which may be part of or separate from the workstation 206. The stimulator 214 delivers electrical signals (such as for pacing or defibrillating the heart) the catheters 234 positioned within the patient. The stimulator 214 is utilized to induce a pacing train of pulses in order to stabilize a refractory period. The pacing train is considered to have “entrained” the heart once it has captured the heart for a predetermined series of beats. Once the heart is entrained, extra stimuli are added to mimic certain capabilities of the heart. The stimulator 214 may drive ventricular protocols through pacing from a ventricular catheter. One reason for ventricular pacing may be to assess the conduction retrograde through the AV node or bypass tract. When assessing conduction retrograde through the AV node, a VAWBK will also be obtained. Another ventricular protocol is the ventricular effective refractory period (VERPS). The stimulator 214 may also be used to induce arrhythmias. For example, during ventricular protocols, ventricular tachycardia or ventricular fibrillation may be induce as an end point. A patient's level of consciousness is assessed while attempts are made at overdrive pacing (if appropriate).

The incoming signals from the patient are passed through sensor module 244 which may perform various signal processing operations upon the incoming signals and/or reroutes the EP signals to the X-ray system 232, ultrasound system 250, IVUS system 252 and workstation 206. The control module 208 manages overall control and operation of the workstation 206. The EP control module 208 receives user inputs through the user interface 210. The EP control module 208 stores data, images and other information in the memory 212. The EP video processor module 216 accesses memory 212 in order to obtain and store various data, signal traces, images and the like. The memory 212 may store diagnostic images, such as ultrasound CT and MR images acquired prior to the procedure. The stored images facilitate pre- and post-procedure analysis for image optimization, manipulation and analysis.

The control module 208 communicates uni-directionally or bi-directionally with video processor module 216 which controls monitors 218 and 220. The monitors 218 and 220 may simply present displayed information as explained hereafter. Optionally, the monitors 218 and 220 may include input buttons for operation by the user to directly enter certain commands and instructions at the monitor 218 and 220. Optionally, the monitors 218 and 220 may represent touch sensitive screens that enable the user to enter information directly by touching active areas of a corresponding monitor 218 and 220.

In the example of FIG. 1, monitors 218 and 220 have been assigned different categories of functions (e.g. real-time monitoring, operations monitoring, documentation monitoring and the like). Monitor 218 presents numerous windows, such as an ablation window, a real-time EP/HD monitoring window and a preprocessing planning window. The monitor 220 displays windows related to operation control, such as an EP/HD recording user interface window, a mapping user interface window and a catheter steering user interface window. The user interface windows allow the operator to enter and change parameters, modes, patient information, values and the like in connection with a particular EP study. Optionally, one of the monitors 218 and 220 may present windows associated with documentation of a particular patient case, such as a case review window, a case reporting window and a case log window. The case-related windows allow the user to review patient history information, as well as current patient information associated with the EP study.

The workstation 206 integrates the display of real-time ultrasound and fluoroscopy images with other EP/HD study information and/or ablation procedure information by utilizing one or more of monitors 218, 220 and 224. For example, the real-time image monitor 224 may present ultrasound images obtained from an ultrasound catheter, while the planning window presents previously acquired CT or MR images. Integrating the ultrasound images into the workstation affords, among other things, an improved standard of care, increased user confidence and shorter procedure time.

Optionally, the real-time image monitor 224 may present ultrasound images as a cine loop, in which a sequence of ultrasound frames is acquired and associated with one or more cardiac cycles. The cine loop of ultrasound images may be repeatedly displayed or frozen. While the real-time image monitor 224 presents the ultrasound images, the monitor 218 simultaneously displays real-time EP or HD signals corresponding to the ultrasound cine loop.

FIG. 2 illustrates an exemplary block diagram of processor module 258 or 268 of the IVUS or ultrasound systems 252 or 250, respectively. The processor module 258, 268 is illustrated conceptually as a collection of modules, but may be implemented utilizing any combination of dedicated hardware boards, DSPs and processors. Alternatively, the modules may be implemented utilizing an off-the-shelf PC with a single processor or multiple processors, with the functional operations distributed between the processors. As a further option, the modules may be implemented utilizing a hybrid configuration in which certain modular functions are performed utilizing dedicated hardware, while the remaining modular functions are performed utilizing an off-the shelf PC and the like. The operations of the modules may be controlled by a local ultrasound controller 302. The modules 306-312 perform operations that may generally be characterized as mid-processor operations.

The processor module 258, 268 receives ultrasound data 304 in one of several forms depending upon the type of probe or catheter. In the embodiment of FIG. 2, the received ultrasound data 304 constitutes I, Q data pairs representing the real and imaginary components associated with each data sample. The I, Q data pairs are provided to a color-flow module 314, a power Doppler module 312, a B-mode module 310, a spectral Doppler module 308 and M-mode module 306. Optionally, other modules may be included such as a strain module, a strain rate module, ARFI module and the like. Each of modules 306-312 process the I, Q data pairs in a corresponding manner to generate color-flow data 324, power Doppler data 322, B-mode data 320, spectral Doppler data 318, M-mode data 316, ARFI module 315, strain data and strain rate data, all of which may be stored in memory 256, 266. The color-flow, power Doppler, B-mode, spectral Doppler, M-mode data, ARFI module 315, strain data and strain rate data 316-325 may be stored as sets of vector data values, where each set defines an individual ultrasound image frame. The vector data values are generally organized based on the polar coordinate system.

The scan converter module 326 reads from memory 256, 266 the vector data values associated with an image frame and converts the set of vector data values to Cartesian coordinates to generate an ultrasound image frame 332 formatted for display. Once the scan converter module 326 generates the ultrasound image frames 332 associated with B-mode data, color-flow data, power Doppler data, ARFI module 315, strain data and strain rate data, and the like, the image frames may be restored in memory 256, 266 or passed over bus 338 to the video processor 260 or 270.

As an example, it may be desired to view a B-mode ultrasound image in real-time on monitor 262 or 272 associated with the ultrasound signals detected by an ultrasound catheter 240 or probe 236, 238 (FIG. 1). To do so, the scan converter module 326 obtains B-mode vector data sets for images stored in memory 256, 266. The B-mode vector data is interpolated where necessary and converted into the X, Y format for video display to produce ultrasound image frames. The scan converted ultrasound image frames are passed to the video processor 260, 270 that maps the video to a grey-scale mapping for video display. The grey-scale map may represent a transfer function of the raw image data to displayed grey levels. Once the video data is mapped to the grey-scale values, the video processor 260, 270 controls the monitor 262, 272 to display the image frame in real-time during a procedure. The B-mode image displayed in real-time is produced from an image frame of data in which each datum indicates the intensity or brightness of a respective pixel in the display. The display image represents the tissue and/or blood flow in a plane through the region of interest being imaged.

The color-flow module 314 may be utilized to provide real-time two-dimensional images of blood velocity in the imaging plane. The frequency of sound waves reflected from the inside of the blood vessels, heart cavities, etc., is shifted in proportion to the velocity of the blood vessels; positively shifted for cells moving toward the transducer and negatively shifted for cells moving away from the transducer. The blood velocity is calculated by measuring the phase shift from firing to firing at a specific range gate. Mean blood velocity from multiple vector positions and multiple range gates along each vector are calculated and a two-dimensional image is made from this information. The color-flow module 314 receives the complex I, Q data pairs from the beamformer 254, 264 (FIG. 1) and processes the I, Q data pairs to calculate the mean blood velocity, variance (representing blood turbulence) and total pre-normalized power for all sample volumes within the operator defined region.

The spectral Doppler module 308 operates upon the I, Q data pairs by integrating (summing) the data pairs over a specified time interval and then sampling the data pairs. The summing interval and the transmission burst length together define the length of the sample volume which is specified by the user at the user interface 257. The spectral Doppler module 308 may utilize a wall filter to reject any clutter in the signal which may correspond to stationery or very slow moving tissue. The filter output is then fed into a spectrum analyzer, which may implement a Fast Fourier Transform over a moving time window of samples. Each FFT power spectrum is compressed and then output by the spectral Doppler module 308 to memory 256. The 2D video processor module 328 then maps the compressed spectral Doppler data to grey scale values for display on the monitor 262 as a single spectral line at a particular time point in the Doppler velocity (frequency) versus a time spectrogram.

The 2D video processor module 328 may combine one or more of the frames generated from the same or different types of ultrasound information. Optionally, the processor module 328 may superimpose an image of one type (e.g., B-mode) on an image of another type (e.g., color Doppler). For example, the 2D video processor modules 328 may combine a B-mode image frame and a color-flow image frame by mapping the B-mode data to a grey map and mapping the color-flow data to a color map for video display. In the final displayed image, the color pixel data is superimposed on the grey scale pixel data to form a single multi-mode image frame 334 that is re-stored in memory 256 or passed over bus 338. Alternatively, the process module 328 may superimpose an image obtained at one point in time with an image obtained at another point in time (e.g., temporal superposition). For example, the processor module 328 may perform image compounding through which two or more images of the same type/mode (but acquired for different spatial regions) are combined to form a larger image.

Successive frames of color-flow and/or B-mode images may be stored as a cine loop in memory 256. The cine loop represents a first in, first out circular image buffer to capture image data that is displayed in real-time to the user. The user may freeze the cine loop by entering a freeze command at the user interface 257. The user interface 257 represents a keyboard and mouse and all other commands associated with ultrasound system user interface.

A 3D processor module 330 is also controlled by user interface 257, 267 and accesses memory 256, 266 to obtain spatially consecutive groups of ultrasound image frames and to generate three dimensional image representation thereof, such as through volume rendering or surface rendering algorithms. The three dimensional images may be generated utilizing various imaging techniques, such as ray-casting, maximum intensity pixel projection and the like.

Returning to FIG. 1, the workstation 206 includes an external video processor module 222 that has access to memory 212 and communicates with the control module 208. The external video processor module 222 controls a separate monitor 224 provided as part of the workstation 206. Monitor 224 is positioned immediately adjacent monitors 218 and 220 in order that all 3 monitors may be reviewed simultaneously by an operator of the workstation 206.

The external video processor module 222 receives video input signals 223, 225, and 227 from the x-ray system 232, the ultrasound system 250 and the IVUS system 252, respectively. The video signals 223, 225 and 227 are directly attached to the video signals used to drive the fluoroscopy monitor 290, ultrasound monitor 272, and IVUS monitor 262, respectively. The external video processor module 222, under direction of the control module 208, affords a comprehensive image management system under which fluoroscopy and ultrasound images may be viewed in real-time at the workstation 206. The external video processor module 222 includes additional video input signals (e.g., such as signal 229) from any standard video source.

By way of example only, the monitor 224 may have a resolution of 1600×1200 pixels and acquire 1 k×1 k images at 72 Hz sampling from multiple video signals 223-229. The video signals 223-227 may be tied directly to VGA outputs of the monitors 290, 272 and 260, which allow images displayed on the fluoroscopy, ultrasound and IVUS systems 232, 250 and 252, to be sent directly to the EP workstation 206 and displayed on the monitor 224 as one of various video input signals. Hence, monitor 224 presents, in real-time, identical information to the information presented on the monitors 290, 272 and/or 260 in the procedure room in real-time.

Alternatively, the external video processor module 222 may be removed and one or more of the links 223, 225, 227 and 229 provided directly to a corresponding input of the monitor 224 (such as indicated by dashed line 231).

Optionally, the fluoroscopy, ultrasound and IVUS images presented on monitors 290, 272 and 262 (and monitor 224) may be synchronized with one another based upon the timing information stored in connection with each fluoroscopy, ultrasound and IVUS image. The timing information may be derived from a system clock, a master oscillator, the cardiac cycle of the patient (as defined within the ECG signals and/or IC or HD signals detected from the patient). Alternatively, the images presented on the fluoroscopy, ultrasound and IVUS monitors 290, 272 and 262 may not be directly synchronized with one another and instead displayed simultaneously in real-time, but in a non-synchronized manner with respect to one another.

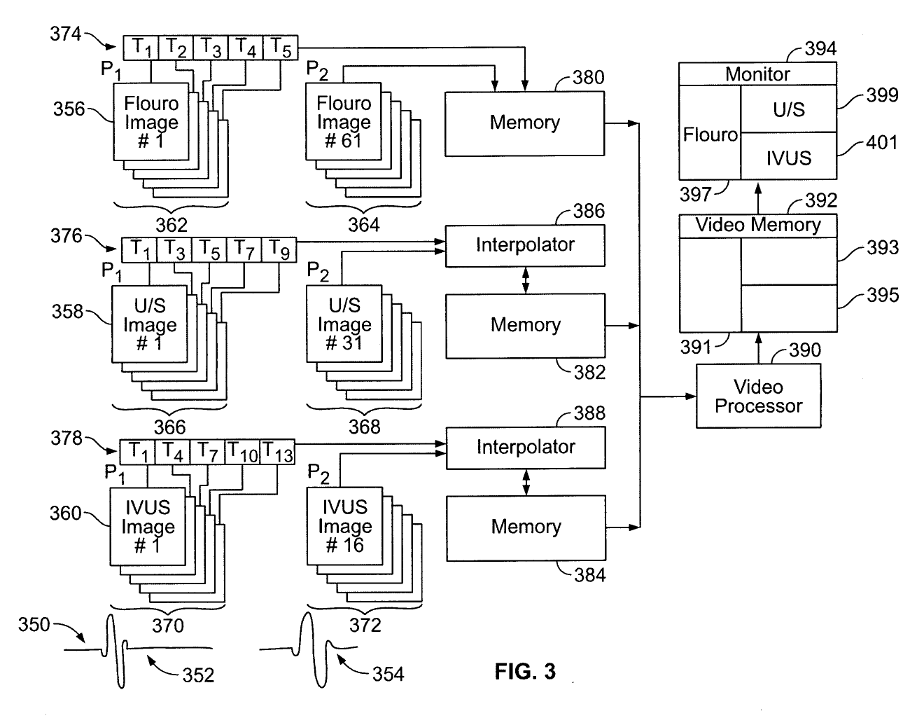

FIG. 3 illustrates a pictorial representation of a processing sequence carried out in connection with image management. The memory and various modules of FIG. 3 may be implemented within the external video processor module 222. In FIG. 3, an ECG signal trace 350 is illustrated for two cardiac cycles 352 and 354. Alternatively, the ECG signal trace 350 may be replaced with an IC or HD signal trace. During each cardiac cycle 352, 354 the x-ray system 232, ultrasound system 250 and IVUS system 260 (FIG. 1) each acquire fluoroscopy and ultrasound data and generate corresponding fluoroscopy, ultrasound and IVUS images 356, 358 and 360, respectively. A first set 362 of fluoroscopy images 356 is acquired during the first cardiac cycle 352, while a second set 364 of fluoroscopy images is acquired during the second cardiac cycle 354. Similarly, first and second sets 366 and 368 of ultrasound images 358 are acquired during the first and second cardiac cycles 352 and 354. First and second sets 370 and 372 of IVUS images 360 are acquired during the first and second cardiac cycles 352 and 354.

The x-ray, ultrasound and IVUS systems 232, 250 and 252 each receive the ECG trace 350 (such as from the sensor module 244 in FIG. 1). Thus, each of the x-ray, ultrasound and IVUS systems 232, 250 and 252 identify a selected, common point in the cardiac cycle (e.g., the P-wave). The commonly selected point in the cardiac cycle is utilized as a common reference point from which all timing calculations are determined. When each fluoroscopy, ultrasound and IVUS image 356, 358 and 360 is obtained, a time stamp is determined. The time stamp may be based on the ECG signal 350 or on synchronization of the clocks of the processors resulting in identical timing of the systems. The time stamp is recorded with each image to identify the precise point during the cardiac cycle at which the image was obtained. With respect to the fluoroscopy images 356, time stamps T1-T5 are illustrated as being correlated to the fluoroscopy images 1-5 in the first set 362.

The fluoroscopy, ultrasound and IVUS imaging systems 232, 250 and 252 may or may not obtain images at an identical or equal rate. The example of FIG. 3 illustrates that fluoroscopy images may be obtained at a rate of approximately 60 frames per cardiac cycle (e.g., when a patient has a heart rate of 60 beats per second, which corresponds to a frame rate of 60 frames per second). The ultrasound and IVUS systems 250 and 252 may obtain images at different rates as well. For example, the ultrasound system may obtain images at a rate of 30 images per second which correlates to 30 images per cardiac cycle in the present example. Images 1-30 are obtained in set 366 during cardiac cycle 352, while images 31-60 are obtained during cardiac cycle 354.

The frame rate of the IVUS system 252 may be, for example, 15 IVUS frames per cardiac cycle. In the example of FIG. 3, set 370 includes IVUS images 1-15 which are obtained during cardiac cycle 352, while images 16-30 are obtained during cardiac cycle 354.

Time stamps T1, T3, T5, T7 and T9 are stored or otherwise correlated with the ultrasound images 1-5 in the image set 366, while time stamps T1, T4, T7, T10 and T13 are stored or otherwise correlated with IVUS images 1-5 in image set 370. The time stamps 374, 376 and 378 are stored in the corresponding memories 380, 382 and 384 with associated images 356, 358 and 360, respectively, in a one-to-one relation. The memories 380, 382 and 384 may be part of memory 212, or alternatively, three separate video memory areas or a common video memory 233. The fluoroscopy images 356 may be loaded directly into the memory 380 by way of example. The ultrasound images 358 and IVUS images 360 may be passed through interpolator modules 386 and 388, respectively before being stored in memories 382 and 384. The interpolator modules 386 and 388 may be part of the external video processor module 222 and may perform temporal interpolation between consecutively acquired images to generate additional “synthetic” images (e.g., images not directly derived from raw echo signals) corresponding to the time stamps for which images based on raw data were not obtained. For example, the interpolator module 386 may generate a synthetic ultrasound image associated with time stamp T2 based on an interpolation between the ultrasound images 1 and 2 which were acquired at time stamps T1 and T3 before and after the time of the synthetic image. The interpolator module 386 repeats this process to generate a number of ultrasound images equal in number to the number of fluoroscopy images 356.

The interpolator module 388 may perform a similar interpolation process, but produce two synthetic images to be inserted between adjacent IVUS images acquired from raw data. For example, interpolator 388 generates synthetic images for times T2 and T3 which are inserted between the images 1 and 2 obtained at time stamps T1 and T4. Interpolator modules 386 and 388 may utilize weighting functions to assign a greater weight to one of the images preceding or succeeding the time at which an interpolated image is being generated. Optionally, multiple consecutive images may be combined (e.g., averaged, from which the synthetic/interpolated image are calculated).

Optionally, the interpolator modules 386 and 388 may not produce synthetic or interpolated images to fill the gaps between acquired images. Instead, the interpolator modules 386 and 388 may simply copy acquired images into the blank image frames. For example, interpolator module 386 may copy U/S image #1 and assign the copy of U/S image #1 to time stamp T2. The interpolator module 388 may copy IVUS image #1 into time stamps T2 and T3, or copy IVUS image #2 (associated with time stamp 4) into the image frames associated with time stamps T3 and T5.

Once the fluoroscopy, ultrasound and IVUS images 356, 358 and 360 are loaded into memories 380, 382 and 384, a video processor 390 (within the video processor module 222) accesses one or more image frames from one or more of the memories 380, 382 and 384 and stores the corresponding image frame or frames in video memory 392, which frame(s) is then reproduced on the monitor 394. By way of example, the video processor 390 may reformat and load fluoroscopy image #1 into memory area 396, ultrasound image #1 into memory area 391, ultrasound image #1 into memory area 393 and IVUS image #1 into memory area 395. The grey scale and/or black and white information in memory areas 391, 393 and 395 are reproduced on the monitor 394 in windows 397, 399 and 401, respectively.

Formatting by the video processor 390 may include changing the resolution of the image, such as from a higher resolution to a lower resolution. For example, the resolution of a fluoroscopy image frame may be 2K by 2K pixels, while the monitor 224 (FIG. 1) may only be able to display a 1K by 1K fluoroscopy image. In this example, the video processor 390 reformats the fluoroscopy image by subtracting every other pixel from the image frame. Alternatively, the video processor 390 may reformat the image frame by applying a smoothing or averaging filter to the pixel values. As another example, the ultrasound image frame may be formatted 1600×1000 pixels, whereas the window into which the image frame is mapped has a resolution of 1200×800. The video processor 390 reformats the image frame by averaging, interpolation, copying of data values, removing data values and the like.

Individual images may be captured as snapshots under the control of the control module 208 and user interface 210. The snapshot images may be passed to a library or other memory 212.

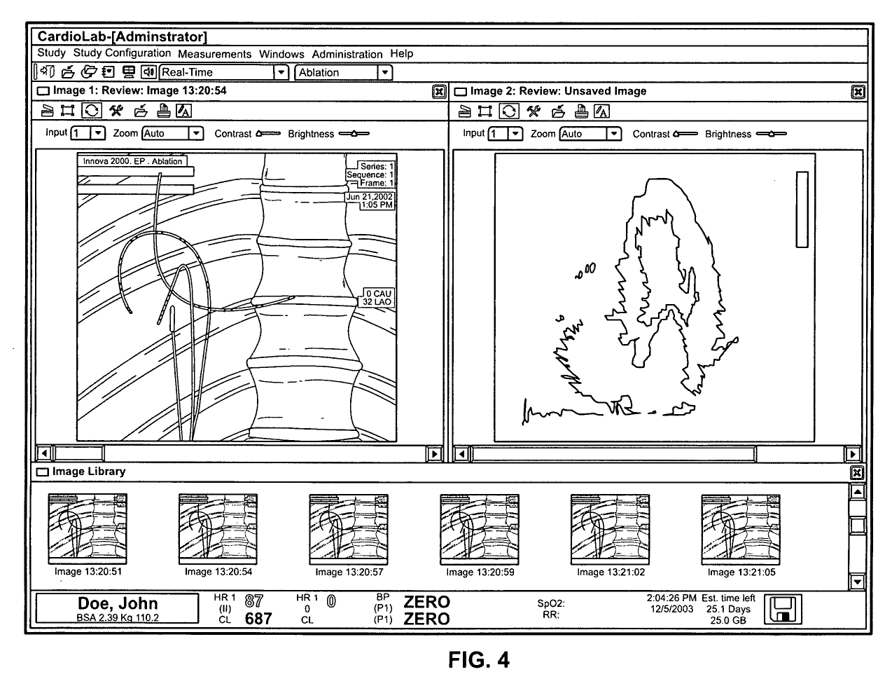

FIG. 4 illustrates a screen shot of an exemplary collection of windows that may be presented on the monitor 394. The screen shot includes a fluoroscopy image window, an ultrasound image window and an image library window. The image library window illustrates a series of previously acquired ultrasound, fluoroscopy or IVUS images, from which the user may select. When the user selects one of the images from the library, it may be illustrated in the image review window, and co-displayed with a real-time image of another modality or saved image of another modality obtained at the same point in the cardiac cycle.

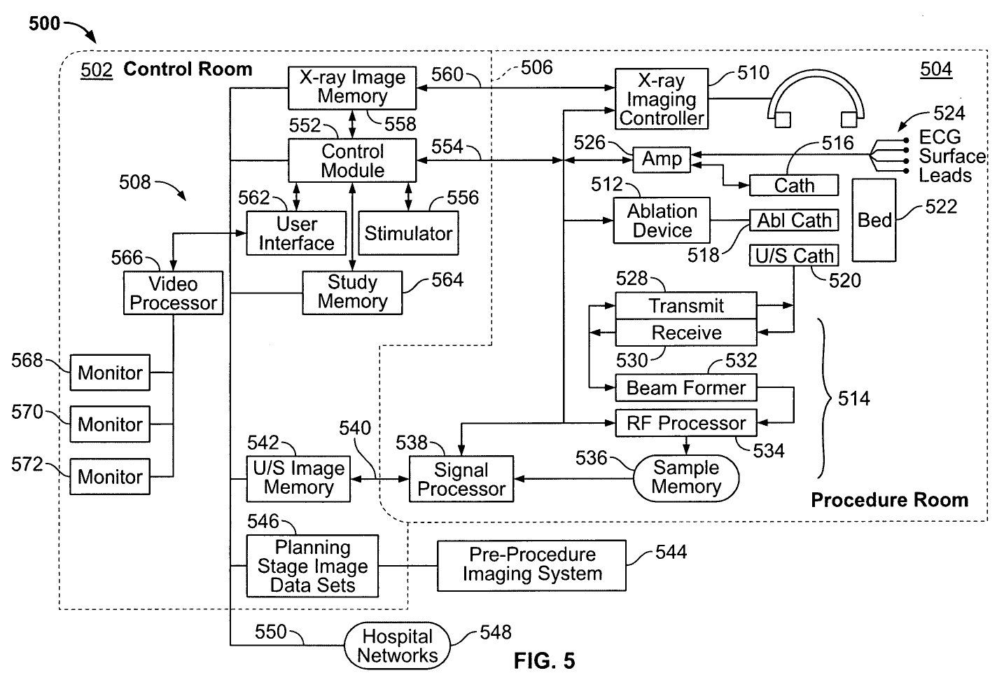

FIG. 5 illustrates a physiology system 500 formed in accordance with an alternative embodiment of the present invention. The system 500 is distributed between a control room 502 and an a procedure room 504 separated by a dividing wall 506. Optionally, the system 500 may be provided all in the procedure room 504. The control includes a workstation 508, while the procedure room includes an x-ray imaging controller 510, an ablation device 512, the backend subsystem 514 of an ultrasound system. EP or HD catheters 516, an ablation catheter 518 and an ultrasound catheter 520 are shown adjacent a bed 522 on which a patient rests during a procedure. ECG surface leads 524 are provided for attachment to the surface of the patient to monitor the ECG signals. An amplifier 526 receives the ECG signals from the surface leads 524 and receives intracardiac and/or hemodynamic signals from the catheters 516. The ablation device 512 controls the ablation catheter 518.

The backend subsystem 514 includes transmit and receive modules 528 and 530 that control transmission and reception of ultrasound signals to and from ultrasound catheter 520. A beamformer 532 is joined to the transmitter and receiver 528 and 530, respectively, and operates in a manner described above to generate RF signals that are passed to an RF processor 534. The RF processor 534 converts the RF signals to I, Q data pairs associated with ultrasound data samples and stores the I, Q data pairs in the sample memory 536. A signal processor module 538 may directly communicate with the RF processor 534 and/or access the sample memory 536 to perform various ultrasound processing functions, such as discussed above in connection with FIG. 2. The signal processor module 538 generates ultrasound images that are passed from the procedure room 504 through the dividing wall 506 along a data transmission link 540 to an ultrasound image memory 542 located in the control room 502 as part of the EP workstation 508. The ultrasound image memory 542 stores sets of image frames as two-dimensional slices or as three dimensional volumes.

A pre-procedure imaging system 544, such as a MR system, CT system, PET, Nuclear System and the like is utilized to obtain medical diagnostic information associated with the patient. The pre-procedure imaging system 544 delivers image data sets to a planning module 546 which stores the image data sets for subsequent processing by the workstation 508. A hospital network 548 is also joined, via a network link 550 to the workstation 508.

The workstation 508 includes a control module 552 that communicates with the x-ray imaging controller 510, ablation device 512, backend subsystem 514 of the ultrasound system over a link 554. The control module 552 also receives, over link 554, ECG and IC signals from the amplifier 526. The control module 552 also delivers stimulus signals over link 554 to the catheter 516. The stimulator 556 is joined to the control module 552 to generate the stimulus signals ultimately delivered to the patient through the catheter 516. The control module 552 communicates with the x-ray image memory 558 which receives x-ray images over link 560 from the x-ray imaging controller 510. A user interface 562 is used to control the workstation 508. The control module 552 stores the ECG signals, IC signals and other procedure related information in the study memory 564. A video processor 566 accesses the x-ray image memory 558, study memory 564, ultrasound image memory 542 and planning module 546 to obtain information and images for display on monitors 568, 570 and 572.

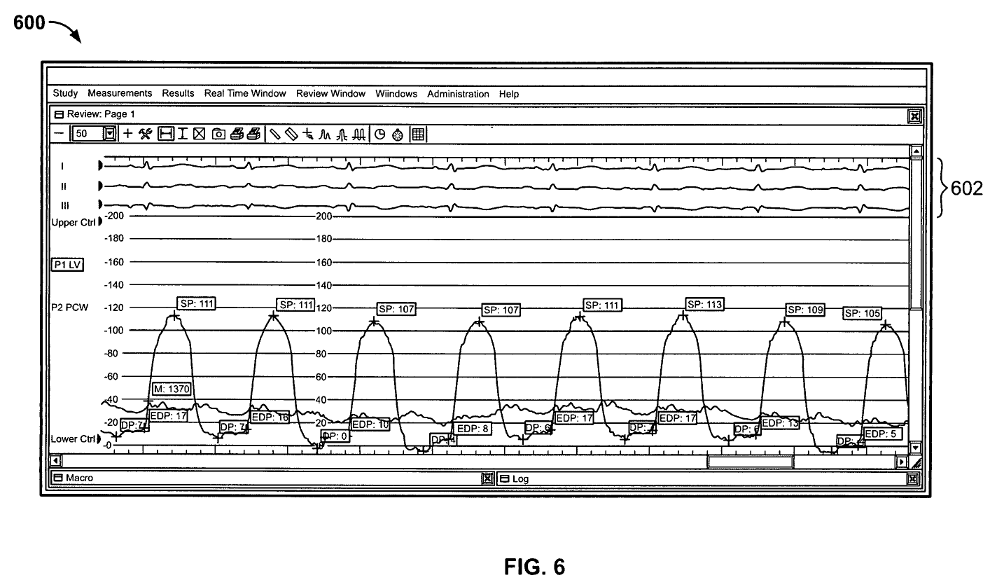

FIG. 6 illustrates a screenshot of an exemplary window presented on one of the monitors of the physiology workstation 206 or 508 of FIG. 1 or 5, respectively. The screenshot of FIG. 6 represents a hemodynamic window 600, including three ECG traces, above a graph plotting the pressure at a particular point within the heart. In the example of FIG. 6, the pressure information is being obtained from an open lumen catheter having an outer end located proximate the mitral valve. The peaks and valleys within the graph represent the diastolic points (DP) and systolic points (SP) in the cardiac cycle. The pressure at each DP and SP is indicated as well. The EDP represents the end diastolic pressure. Along the bottom of the graph are a series of time stamps identifying the time (relative to the system clock) at which each pressure point was measured. The upper and lower controls (UpperCtrl and LowerCtrl) may be adjusted by the operator to adjust the dynamic range over which the pressure is measured.

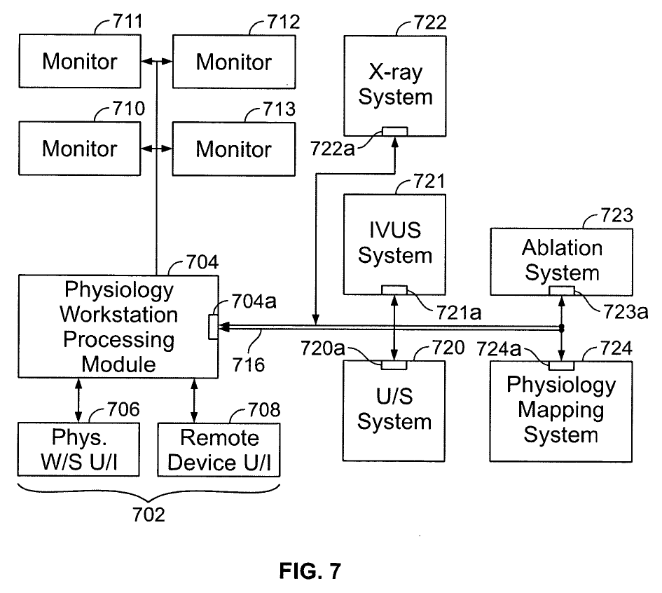

FIG. 7 illustrates a block diagram of an alternative embodiment in which remote control is provided for various systems and devices. In FIG. 7, a physiology workstation 702 (e.g. EP or H. D. workstation) and includes a physiology workstation processing module 704 that communicates with, and is controlled by, a physiology workstation user interface 706. The physiology workstation 702 may be located in a new separate room (e.g. a control room) remote from the systems 720-724. Alternatively, the physiology workstation 702 may be located in the same room as the systems 720-724. A remote device user interface 708 also communicates with the physiology workstation processing module 704. The monitors 710-713 are joined to the physiology workstation processing module 704 to illustrate the various information, images, signals and the like explained above. A link 716 is maintained between the physiology workstation processing module 704 and various remote devices, such ultrasound system 720, IVUS system 721, x-ray system 722, ablation system 723 and physiology mapping system 724. The systems 720-724 may each include the associated types of acquisition apparatus (e.g. catheters, probes, C-arm, coils and the like, as well as monitors and user interfaces).

The link 716 may include one or more links connected to each of the systems 720-724. For example, the link 716 may include a single serial or parallel line directly extending from the remote device user interface 70821 of the systems 720-724, and attached thereto, at a user interface input. Alternatively or in addition, link 716 may include a data bus conveying serial or parallel data between the processors within module 704 and one or more of systems 720-724 (e.g. ECG data, EP data, HD data, image frames and the like). The link 716 may also include one or more video cables extending between a video output (e.g. VGA) at one of systems 720-724 and a video input at one or more of monitors 710-713.

Optionally, the link 716 may constitute a network connection, such as supporting an Internet protocol (IP) or the transmission control protocol (TCP), or other protocols. The data may be transmitted over link 716 as raw ultrasound or x-ray data, formatted in the Hypertext markup (HTML) language, and the like. Optionally, the link 716 may be constructed as a local area network configuration, a client/server configuration, an intranet configuration, a file sharing configuration and the like. Communications modules 704a and 720a-724a would be provided at each of the module 704 and systems 720-724 configured in accordance with the appropriate configuration. The communications modules 704a and 720a-724a may represent USB ports, while the link 716 represents a USB cable. Alternatively, the communications modules 704a and 720a-724a may represent serial or parallel connectors, HSSDC connectors, Fiber Channel connectors and the like, while the link 716 represents the corresponding type of communications medium. Alternatively, the link 716 may be wireless (e.g., RF, Bluetooth, etc.).

The remote device user interface 708 may be used to control the operation of one or more of the systems 720-724. For example, the remote device user interface seven OA may be used to enter system parameters, settings, modes and the like. The remote device user interface 708 permits the operator of the physiology workstation 702 to remotely control the operation, and remotely adjust the settings, modes and parameters, of one or more of the systems 720-724. The remote device user interface 708 improves workflow within the procedure room, increases productivity of an EP or HD team in the procedure room and end the review room, and decreases the overall procedure duration.

By way of example, when the remote device user interface 708 is used in connection with control of the ultrasound system 720 or IVUS system 721, the remote operator may be afforded the ability to change a modes, adjust the gain of the ultrasound probe or catheter, freeze select images on the monitor at the physiology workstation 702 and the monitor at the ultrasound system 720, and the like. Optionally, the remote device user interface 708 may constitute a dedicated keyboard identical to a keyboard provided with one of systems 720-724.

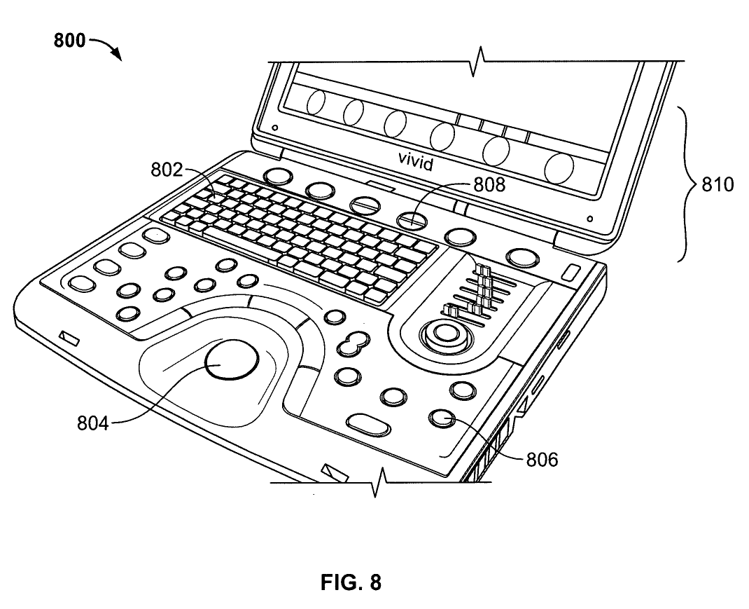

FIG. 8 illustrates an exemplary remote device user interface 708 constructed one to substantially resemble the keyboard of an ultrasound system. the keyboard 800 includes a keypad 802, a trackball 804, various dedicated buttons 806 related to particular ultrasound modes and settings. The keyboard 800 also includes soft keys 808, the function of which changes depending upon the mode of operation. The selected function of each SoftKey 808 is indicated on the lower portion of the monitor and the SoftKey function area 810.

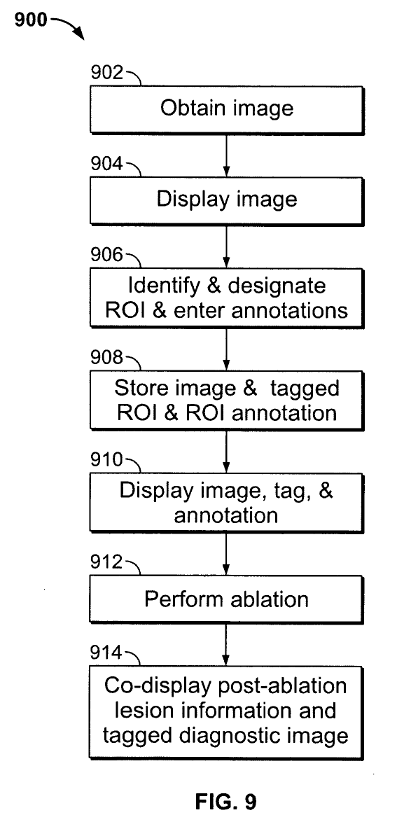

FIG. 9 illustrates a processing sequence that may be carried out by the physiology workstation 206 (FIG. 1) to provide a region tagging feature. Region tagging 900 permits an operator of the physiology workstation 206 to tag regions/points of interest in images obtained by various diagnostic systems (e.g., ultrasound, IVUS, x-ray, CT, MRI, NM, PET, physiology mapping and the like. At 902 physiology workstation 206 obtains one or more diagnostic images from one or more of the IVUS system 260, ultrasound system 250 and x-ray system 232. At 904 the diagnostic image is displayed on one or more of monitors 218, 220, 224, 262, 272 and 290. The control module 208 may also passes the diagnostic image over network 209 and link 213 to a remote network site 211, or a consultant may be located.

The operator of the physiology workstation 206 utilizes the user interface 210 to identify and to tag or designate a region or point of interest (ROI) within the diagnostic image (e.g. a lesion, a pulmonary vein, the mitral valve and the like). Optionally, a consultant at the remote network site 211 may identify and tag or designate the ROI. By way of example, the ROI may be designated by moving a cursor, at a review or physiology workstation, to a point within the diagnostic image through manipulating a trackball or mouse or arrow keys, and then pressing a key or mouse to select the position of the cursor. Alternatively, a trackball or mouse may be used to draw a boundary around the ROI by selecting a series of points as the cursor is moved around the ROI. The ROI may represent a point or an area having a predefined contour and dimension which may be adjusted dynamically during analysis of the diagnostic image, or pre-procedure based on an individual user's preferences. In addition, the user at the physiology workstation 206 or a consultant at the remote network site 211 may add annotations proximate the ROI, such as a label identifying the region of interest. Optionally, the annotation information may include comments and notes that are not directly imposed upon the diagnostic image, but instead are attached to the file containing the diagnostic image as a separate text file.

At 908, the diagnostic image, ROI tag and annotations information are stored in memory 212 and/or video memory 233. When a consultant at the remote network site 211 receives diagnostic images, and adds ROI tags and annotations, a tag image file is created including the diagnostic image, ROI tags and annotation information. The tag image file is returned over link 213 and network 209 to the control module 208, which stores the tag image file in memory 212 and/or video memory 233. At 910, the control module 208 accesses the memory 212 or video memory 233 to obtain and display the combination of the diagnostic image, tag and annotations information on one or more of the various monitors 218, 220, 224, 262, 272 and 290. Following 910, the operation may stop.

Alternatively, following 910, processing may continue to 912, at which an ablation procedures performed such as on the region of interest. Following the ablation procedure, the tag the diagnostic image may be retrieved from memory and redisplayed. The tag the diagnostic image may be presented alone or co-displayed (at 914) with post ablation lesion information. The post ablation lesion information may represent a computer estimation of the area exposed to ablation, a direct visual observation of the region of elated (such as obtained through IVUS imaging) and the like. For example, the post ablation lesion information may be presented as a two-dimensional image (actual or computer generated) similar in format and scale to the diagnostic image obtained at 902. For example, when the diagnostic image represents a two-dimensional B-mode ultrasound image formatted as a sector scan, the post ablation lesion information may be presented in a similar format to facilitate review.

In accordance with the procedure of FIG. 9, the physiology workstation 206 affords the ability to visually tag any point in ultrasound image, either from the procedure room, a review room or a remote network site. Candidate ablation points may be tagged by either an operator or a remote consultant, thereby affording a high standard of care, increased staff confidence, increased procedure speed and minimal interference of the consultant's time. The visual tags may be placed anywhere on the diagnostic image and may be marked by the system operator or by a remote operator with a remote keyboard interface. Tags marked by the system operator or a remote operator may be displayed on multiple monitors, such as the ultrasound monitor and a monitor at the physiology workstation 206, as well as at a remote monitoring suite.

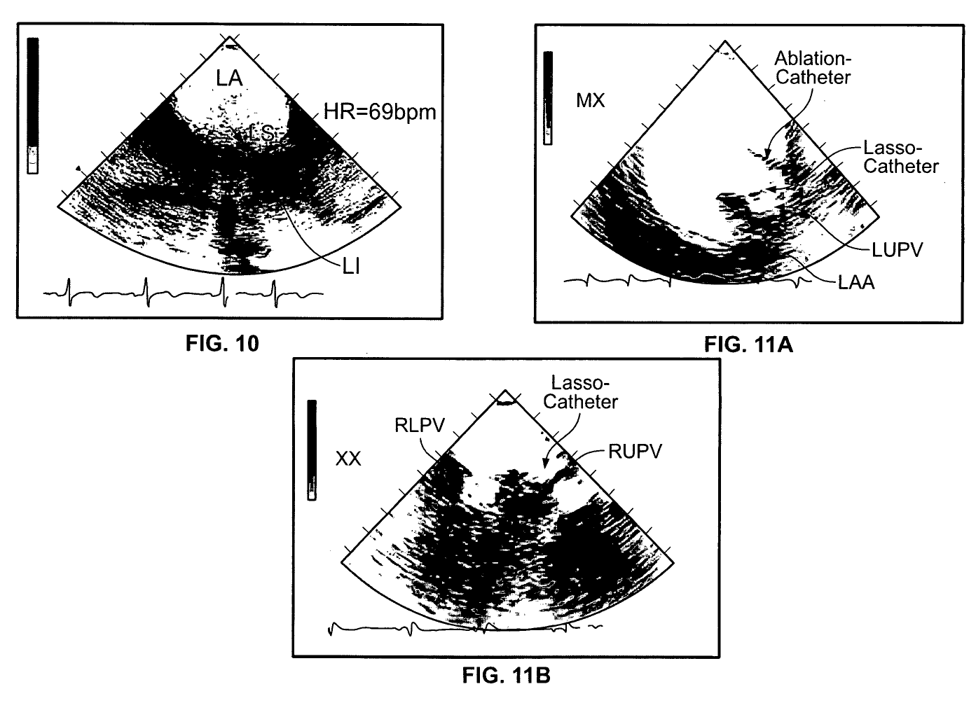

FIGS. 10A and 10B illustrate images acquired by an echocardiography (ICE) catheter. The ICE catheter is positioned in the right atrium proximate one side of the right atrium and is directed toward the opposite side of the right atrium. The images indicate a “Coil” representing, in the example, an ablation catheter coil. The indicia TV denotes the tricuspid valve. The area denoted ICE presents a second echocardiography catheter inserted in the right atrium. In FIG. 10A the coil is located against the wall of the right atrium, while in FIG. 10B the coil is not in contact with the wall of the atrium.

FIG. 11 illustrates an ultrasound image obtained by an ICE catheter located on the right side of the inter-atrial septum positioned to image the left pulmonary veins. The indicia LA denotes the left atrium, while the indicia LS and LI denote the left superior and left inferior pulmonary veins, respectively.

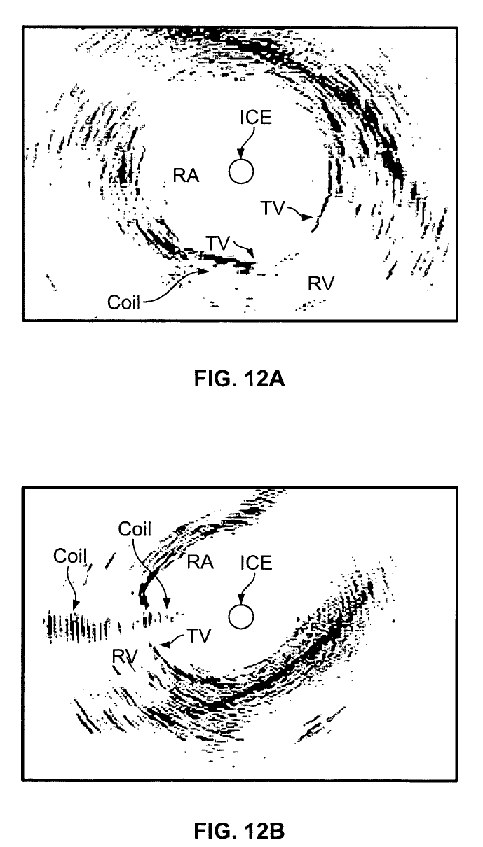

FIGS. 12A and 12B represent ultrasound images obtained by an ICE catheter. In FIG. 12A, the indicial LAA denotes the left atrial appendage, and LUPV denotes the left upper pulmonary vein. An ablation catheter and lasso catheter are also present in the left atrium. In FIG. 12A, the indicia RUPV and RLPV denote the right upper and lower pulmonary veins, respectively. A lasso catheter is also present in the left atrium. The ICE catheter, ablation catheter and lasso catheter were inserted into the left atrium from the right atrium by puncturing the fossa ovalis.

The figures illustrate diagrams of the functional blocks of various. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed imaging software package, and the like.

What is claimed is:

1. A physiology workstation, comprising:
a physiology input configured to receive physiology signals from at least one of an intracardiac (IC) catheter, a hemodynamic catheter and surface ECG leads provided on a subject, the physiology signals being obtained during a procedure;
a video input configured to receive image frames, in real-time during the procedure, the image frames containing diagnostic information representative of data samples obtained from the subject during the procedure;
a physiology control module controlling physiology operations based on user inputs; and
a display module controlled by the physiology control module, the display module displaying the physiology signals and the image frames simultaneously, in real-time, during the procedure.

2. The workstation of claim 1, further comprising a video processor module formatting the physiology signals into a display format.

3. The workstation of claim 1, further comprising an physiology video processor and an external video processor receiving and controlling display of the physiology signals and image frames, respectively.

4. The workstation of claim 1, wherein the image frames include at least one of ultrasound images obtained from a surface ultrasound probe, intravenous ultrasound images obtained from an ultrasound catheter and fluoroscopy images obtained from a fluoroscopy system.

5. The workstation of claim 1, wherein the physiology signals and image frames are displayed in a synchronized manner based on one of a system clock and a cardiac cycle of the subject.

6. The workstation of claim 1, wherein the physiology signals and image frames are displayed in a non-synchronized manner.

7. The workstation of claim 1, wherein the display module includes first and second monitors, the first monitor displaying the physiology signals and the second monitor displaying the image frames both in real-time during the procedure side-by-side for viewing by the operator of the physiology workstation.

8. The workstation of claim 1, wherein the display module is located in a control room remote from a procedure room in which the subject is located.

9. The workstation of claim 1, wherein the video processor module performs interpolation between consecutive image frames to form synthetic frames, the display module displaying the image frames and synthetic frames in an interleaved manner.

10. The workstation of claim 1, wherein the video input receives image frames at a first frame rate, the video processor module processing the image frames to present the image frames on the display module at a second frame rate that differs from the first frame rate.

11. The workstation of claim 1, wherein the video input receives image frames with the diagnostic information formatted with a first resolution, the video processor module processing the image frames to present the diagnostic information on the display module at a second resolution that differs from the first resolution.

12. The workstation of claim 1, further comprising memory storing the image frames in an image library.

13. The workstation of claim 1, further comprising a user interface offer the operator a snapshot function, the physiology control module obtaining a single snapshot image frame from the image frames received at the video input when the snapshot function is selected.

14. The workstation of claim 1, further comprising a user interface offer the operator a snapshot function, the control module storing a single snapshot image frame from the image frames received at the video input when the snapshot function is selected.

15. A method for managing a physiology workstation, comprising:
receiving, at a physiology workstation, physiology signals from at least one of an intracardiac (IC) catheter, a hemodynamic catheter and surface ECG leads provided on the subject, the physiology signals being obtained during a procedure;
receiving, at the workstation, image frames, in real-time during the procedure, the image frames containing diagnostic information representative of data samples obtained from the subject during the procedure;
controlling physiology operations based on user inputs; and
displaying the physiology signals and the image frames simultaneously, in real-time, during the procedure at the workstation.

16. The method of claim 15, further comprising formatting the physiology signals into a display format.

17. The method of claim 15, wherein the image frames include at least one of ultrasound images obtained from a surface ultrasound probe, intravenous ultrasound images obtained from an ultrasound catheter and fluoroscopy images obtained from a fluoroscopy system.

18. The method of claim 15, further comprising synchronizing the physiology signals and image frames and displaying the physiology signals and image frames in a synchronized manner based on one of a system clock and a cardiac cycle of the subject.

19. The method of claim 15, wherein the physiology signals and image frames are displayed in a non-synchronized manner.

20. The method of claim 15, further comprising displaying the physiology signals on a first monitor and displaying the image frames on a second monitor both in real-time during the procedure side-by-side for viewing by the operator of the workstation.

21. The method of claim 15, further comprising interpolating between consecutive image frames to form synthetic frames, and displaying the image frames and synthetic frames in an interleaved manner.

22. The method of claim 15, wherein the image frames are received at a first frame rate, the method further comprising processing the image frames to display the image frames at a second frame rate that differs from the first frame rate.

23. The method of claim 15, wherein the image frames are received with the diagnostic information formatted with a first resolution, the method further comprising processing the image frames to display the diagnostic information at a second resolution that differs from the first resolution.

24. The method of claim 15, further comprising storing the image frames in an image library.

25. The method of claim 15, further comprising offering the operator a snapshot function, and obtaining a single snapshot image frame from the image frames received when the snapshot function is selected.

26. The method of claim 15, further comprising offering the operator a snapshot function, and storing a single snapshot image frame from the image frames received when the snapshot function is selected.
